(19) United States
(12) Patent Application Publication
(10) Pub. No.: US 2010/0075395 A1
(43) Pub. Date: Mar. 25, 2010

(54) MICROBIAL PROCESS FOR PRODUCTION OF ENZYMES

(75) Inventors: Stahmann Klaus-Peter, Annahuette (DE); Susanne Nieland, Schipkau (DE); Anne Wuttke, Reichenbach (DE)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Fachhochschule Lausitz, Senftenberg (DE)

(21) Appl. No.: 12/517,766

(22) PCT Filed: Oct. 29, 2007
(86) PCT No.: PCT/EP2007/009378

§ 371 Date: Nov. 13, 2009

(30) Foreign Application Priority Data
Dec. 4, 2006 (DE) 10 2006 057 724.8

(51) Int. Cl. C12N 9/20; C12N 9/26; C12N 9/50

(52) U.S. Cl. 435/198; 435/201; 435/219

(57) ABSTRACT

The present invention relates to a suspension culture process for producing an enzyme, comprising the steps of providing a minimal medium, which contains deionised water, mineral salts and an organic carbon source, in a vessel; inoculating the minimal medium with a fungus; incubating the minimal medium at a pH value of 1-4 for a period of time which is sufficient for the fungus to be optically visible in the minimal medium, and obtaining the enzyme, wherein the minimal medium and the vessel are not sterilised. In addition, the invention relates to an enzyme obtainable by the process according to the invention.

### FIELD OF THE INVENTION

The present invention relates to a submerged culture process for producing an enzyme and an enzyme obtainable by this process.

### BACKGROUND OF THE INVENTION

Today, there are known about 5,000 chemical reactions which can be catalyzed by enzymes. For each of these reactions, several, in part over 100 different enzymes with different properties, have been described. Moreover, genetic engineering makes “protein engineering” possible. Nonetheless, only about 10 enzymes are being used in industry at present, e.g. glucose isomerase. There are examples of chemical processes which could be substituted by enzymatic methods, i.e. the functional efficiency was demonstrated in the laboratory. Nevertheless, no transition to production has been made because the price for the enzyme is too high. This applies, for example, to bleaching in the paper manufacture, which is possible with laccases, or to the transesterification of polyurethane precursors, which can take place with lipases.

With the exception of molecular-biological research or medical diagnostics, where more than 100 enzymes, e.g. restriction endonucleases, are being used, the high price for the manufacture and obtaining of enzymes often plays the decisive role.

By heterologous expression of genes in established hosts, e.g. in bacteria such as Escherichia coli or Bacillus spec., but also in fungi, e.g. Pichia pastoris, almost every enzyme can be manufactured with high productivity, i.e. with a high proportion of active protein per biomass.

However, the costs for the manufacture are high because all hosts known to date must be cultivated in previously sterilised equipment and media. The sterile technology necessary for this is a main disadvantage of most of the conventional microbial processes. Some processes never came to use because contamination could not be controlled. An example for that is ethanol production using Zymomonas mobilis.

Sterile technology means not only high investment costs for autoclaves and steam generators as well as temperature-stable and pressure-resistant containers, pipelines and valves made of steel, but also high operating costs in form of energy as well as time for heating and cooling.

Because of these high costs, the space-time yields must be maximised by way of high growth rates and high final cell densities. This requirement entails a chain of consequences, which, in turn, result in high costs. Consequently, complex media with expensive constituents, e.g. yeast extract, are used first. In addition, for optimal gas exchange, stirrers must be operated at high speed. This costs double the energy because it additional cooling is required. Moreover, in the case of recombinant methods, the complex media do not permit the use of complementation markers, e.g. for amino acid auxotrophies. Instead, for example for the production of recombinant enzymes with Bacillus spec., dominant markers, i.e. resistance genes for antibiotics, are used. The latter represent a further cost factor. In this case, it is a disadvantage that the use of antibiotics results in limitations, e.g. when the products are used for nutrition.

The object of the present invention is therefore based on providing a process for producing an enzyme, which overcomes the disadvantages summarised above.

While conventional processes require investment costs in the tens of millions, the costs for carrying out the invention lie in the order of magnitude of an agricultural vehicle, e.g. of a tractor. This makes a new independent occupation possible, that of the “enzyme farmer”.

### SUMMARY OF THE INVENTION

The present invention relates to a submerged culture process for producing an enzyme, comprising the steps of
a) providing a minimal medium comprising deionised water, mineral salts and an organic carbon source, in a vessel,
b) inoculating the minimal medium with a fungus,
c) incubating the minimal medium at a pH value of 1-4 for a period of time which is sufficient for the fungus to be optically visible in the minimal medium, and
d) obtaining the enzyme,
   wherein the minimal medium and the vessel are not sterilised.

The invention also comprises an enzyme obtainable by the process according to the invention.

### BRIEF DESCRIPTION OF THE FIGURES

FIG. 1 shows the growth of the Phialemonium spec. and of the Acremonium strictum isolate AW02 on an agar plate comprising a rich medium consisting of 10 g/l glucose and yeast extract, which was solidified with 20 g/l of agar. An entire agar plate is shown on the left, and an enlarged sectional view of the aerial mycelium on the right.

FIG. 2 shows a fluorescence microscopy image of the Phialemonium spec. and of the Acremonium strictum isolate AW02 after staining with Nile red. The hydrophobic fluorescent dye preferably incorporates itself into lipid droplets, which serve the cells possibly as reserve substance. A large droplet has a diameter of approximately 5 μm.

FIG. 3 shows a phase contrast image of the culture broth after 3 days of growth at 34° C. and 120 rpm in minimal medium with 50 g/l glucose. To be seen are predominantly spores.

FIG. 4 shows the lipase activity in a culture in a large open vessel. After stirring the culture using a concrete agitator, a sample was withdrawn and homogenised by passing it through a French press. The homogenate was used in a continuous colorimetric lipase assay. In the above assay, the hydrolysis of para-nitrophenyl palmitate was measured at 405 nm. One unit (U) was equivalent to 1 μmol of para-nitrophenol released per minute.

FIG. 5 shows a simple culture system for a lipase producer. 100 l were prepared with 20 g/l soya bean oil, as described in Table 4. The culture was mixed by means of compressed air via a frit and maintained at 21° C. Over a period of 14 days no contamination with other microorganisms was microscopically detectable. However, only slow growth was observed. At the end of the experiment, a muddy mycelium mass was found on the bottom of the vessel.

FIG. 6 shows a vessel in the form of a large vessel having a capacity of 100 l culture medium. There is provided a device for introducing sterile-filtered air. A second identical vessel was cut vertically into six segments. These segments were used to facilitate the harvesting of the biofilm. After inoculating the medium with a spore suspension, the growing mycelium colonised the surface of the segments and formed a biofilm. Approximately 90% of the lipase activity could be harvested by removing, draining, and scraping off the segments.

### DETAILED DESCRIPTION OF THE INVENTION

The present invention first relates to a submerged culture process for producing an enzyme, comprising the steps of
a) providing a minimal medium comprising deionised water, mineral salts and an organic carbon source, in a vessel,
b) inoculating the minimal medium with a fungus,
c) incubating the minimal medium at a pH value of 1-4 for a period of time which is sufficient for the fungus to be optically visible in the minimal medium, and
d) obtaining the enzyme,
   wherein the minimal medium and the vessel are not sterilised.

Essential for the invention is the isolation of a microorganism which will reliably establish itself on a non-sterilised medium. Concrete conditions were set as selective conditions. First, a minimal medium having nitrate as nitrogen source was used. Furthermore, a carbon source was used, with emulsified vegetable fat as only carbon and energy source proving to be advantageous. In addition, the initial pH value was adjusted to an acid range of from 1 to 4, with a pH value of 3 being particularly suitable. An incubation temperature of 35° C. has also proven to be advantageous.

In step c), the fungus can initially settle visibly on the vessel wall as a biofilm. If the fungus is grown in suspension culture in a shake flask or baffled flask, the minimal medium becomes cloudy as a result of the fungus growth. This optical turbidity of the medium, which is visible to the naked eye, is also a sign that the fungus was grown for a sufficiently long period of time.

In step d), the enzyme can be obtained from the biofilm or from the minimal medium (culture broth).

With a microorganism isolated in such a way, the simplest conceivable submerged culture process was established, tested, and used for the production of lipase.

In an advantageous embodiment, the enzyme is selected from the group consisting of lipase, amylase and protease. These belong to the class of the extracellular hydrolases. Lipases cleave triglycerides into fatty acids and glycerol, which can then be taken up by the microorganism into the cell. Amylases cleave alpha-1,4-linked glucose chains. So-called endoglucanases release oligomers, while exoglucanases usually split off maltose. Here, too, the decomposition is used to enable the microorganism to incorporate the decomposition products into the energy metabolism and the anabolism. The protease finally hydrolyses proteins into peptides, for which there are highly efficient importers in funguses. Intracellularly, the breakdown into amino acids and, as required, the decomposition into keto acids, or the activation for the protein biosynthesis then takes place.

It is further advantageous for the production of lipase to use a fat as carbon source, in particular vegetable triglycerides, such as for example soya oil, sunflower oil or rapeseed oil. Only few organisms can use those as the only source of carbon and energy. Soya oil has the particular advantage that only few funguses can use this fat quickly and effectively as single carbon source. Soya oil thus also acts as selection means, in order to limit the growth of other, undesired microorganisms. For the production of amylase in particular starch is suitable as carbon source. In particular gelatin can be used to produce a protease enzyme.

It has proven to be particularly advantageous to use nitrate as nitrogen source. Nitrate is inexpensive and excludes microorganisms as contaminants of the process, which can not reduce nitrate.

In a preferred embodiment of the invention, the minimal medium comprises, relative to one litre of water
approximately 0.5 to 10 g KNO₃
approximately 0.5 to 5 g KH₂PO₄
approximately 0.2 to 0.75 g MgSO₄×7H₂O
approximately 0.2 to 0.75 g FeSO₄×7H₂O
approximately 0.2 to 0.75 g ZnSO₄×5H₂O
approximately 0.01 to 0.05 g CuSO₄×5H₂O
approximately 0.01 to 0.05 g MnCl₂×4H₂O and
approximately 5 to 100 ml soya oil.

Most preferably, the minimal medium comprises, relative to one litre of water
approximately 1.5 g KNO₃
approximately 1.5 g KH₂PO₄
approximately 0.5 g MgSO₄×7H₂O
approximately 0.5 g FeSO₄×7H₂O
approximately 0.5 g ZnSO₄×5H₂O
approximately 0.02 g CuSO₄×5H₂O
approximately 0.02 g MnCl₂×4H₂O and
approximately 18 ml soya oil.

In order not to introduce any growth inhibiting substances via the water, deionised water must be used.

It is further preferred to select the fungus for the enzyme production from the group consisting of deuteromycetes and anamorphs of the ascomycetes.

The fungi Phialemonium spec., Acremonium strictum, Aspergillus spec., Botrytis cinerea or Trichoderma spec. appear to be particularly suitable.

The process according to the invention is carried out at an acid pH value of between approximately pH 1 and pH 4. An initial pH value of 3 has proven to be particularly advantageous for the production of lipase on the basis of soya oil. When in particular the fungus Aspergillus nidulans is used for the production of lipase, an even lower pH value of approximately 2.5 may prove particularly suitable.

Since the fungus is grown in a minimal medium on a mineral salt basis and a carbon source which has to be hydrolysed first, it has been found that the incubation time can be up to 14 days.

Enzyme production can be significantly increased if the process according to the invention is performed at a temperature of between approximately 20° C. and approximately 45° C., preferably at 20° C. to approximately 35° C., more preferably at approximately 21° C. Particularly preferred is a temperature range above room temperature, most suitably at approximately 35° C.

As enzyme production by the funguses preferably takes place in an aerobic environment, in a further embodiment it is preferable to aerate the minimal medium. For this purpose the aeration via a sterile filter and a frit is particularly suitable.

Even though the process can be carried out to the greatest extent under non-sterile conditions and thus extremely cost-effectively, it has proven advantageous to use sterile conditions for the inoculum culture with which the minimal medium is inoculated. This ensures that the minimal medium is inoculated with a pure fungus culture, which is not contaminated by bacteria or other undesirable microorganisms.

In order to simplify obtaining the enzyme from the process according to the invention, segments having a large surface and being easy to remove and easy to clean are introduced into the boiler or the large vessel, on which segments the fungus grows as biofilm and can subsequently be easily harvested from the culture and be further processed. Preferably, those funguses are hence used, in which the desired hydrolases are not freely diffusible but rather cell-associated.

The volume of the minimal medium is preferably approximately 1 litre to approximately 3,500 m³. Suitable is a volume of approximately 100 litres. A volume of approximately 350 litres has proven particularly suitable. In the simplest case, a large vessel or, alternatively, a boiler or a barrel which is inoculated with a spore suspension can be used as culture vessel.

A further object of this invention is an enzyme, preferably a lipase, amylase or protease, obtainable by the process according to the invention.

### Preferred Embodiments and Detailed Examples

The following examples illustrate the invention in more detail. They are provided as examples and are not intended to limit the scope of protection of the invention.

### Example 1

Isolation of a Suitable Microorganism

Samples from different compost heaps were streaked onto agar plates having the following medium composition.

| | | |
|---|---|---|
| KNO₃ | 1.5 | g |
| KH₂PO₄ | 1.5 | g |
| MgSO₄ × 7H₂O | 0.5 | g |
| FeSO₄ × 7H₂O | 0.5 | mg |
| ZnSO₄ × 7H₂O | 0.5 | mg |
| CuSO₄ × 5H₂O | 0.02 | mg |
| MnCl₂ × 4H₂O | 0.02 | mg |
| Soya oil | 18 | ml |
| Agar | 20 | g |
| H₂O, deionised | ad 1 | litre |

pH value: 3

The soya oil was emulsified prior to coating the plates. Initially, the incubation was carried out at room temperature. Subsequently, the temperature was gradually increased to 35° C. With the secondary inoculation onto new plates, macroscopically different colonies were separated. The microorganism isolated in this way was designated as AW02 and identified as Acremonium strictum by the German Collection of Microorganisms and Cell Cultures (DSMZ) in Braunschweig. A second analysis has shown that it is Phialemonium spec.

The mycelium of Acremonium strictum was described as very delicate. The diameter of the hyphae was approximately 1-1.5 μm. The conidia carriers were short and rare. The phialides were monophialidic, non-chromophilic, arranged laterally on undifferentiated hyphae, up to 25 μm long, and were present mostly directly on the aerial mycelium. The conidia were cylindrical/ellipsoid, having a size of approximately 4-5×1.6 μm, and they were arranged in a small slimy cup. No chlamydospores could be detected.

Acremonium strictum has been described in the scientific literature as lipase producer. Okeke and Okolo, (1990), Biotechnology Letters 12:747-750, however, used a rich medium with 10 g/l peptone for the culture. In this publication, the execution of a standard lipase assay is also described. The lipase activity could be measured by a titration procedure. The assay mixture contained 5 ml of an enzyme solution and 5 ml citrate/Na₂HPO₄ buffer (pH 7.5) with 0.05 M CaCl₂ and 3% (v/v) Tween 80. The reaction was carried out under stirring at 35° C. for 30 min. It was terminated by the addition of 10 ml acetone/ethanol mixture (1:1, v/v). The amount of fatty acids released was titrated with 0.05 M KOH using 0.3 ml 1% phenolphthalein solution as an indicator. An enzyme solution boiled for 3 min was used as a control. With this assay it was for example possible to measure a lipase activity of greater than 400 U/ml after growth on xylose.

According to the Genetic Engineering Safety Ordinance, Phialemonium spec. and Acremonium strictum belong to the lowest risk group 1. Organisms which are characterised by experimentally proven and long-term safe application, thus not posing any risk to humans, animals or to the environment, belong to this group.

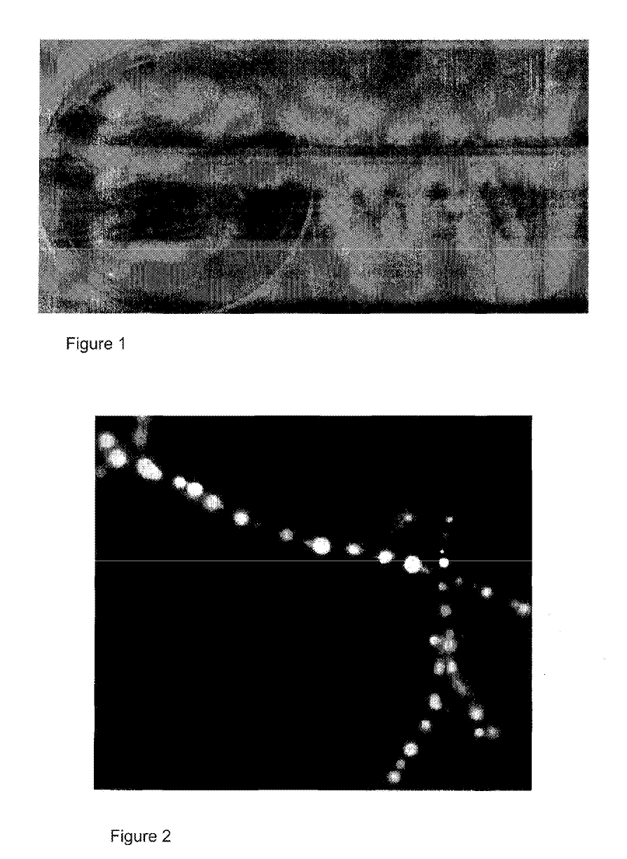

The microorganism Phialemonium spec. and Acremonium strictum AW02, respectively, grew with white substrate mycelium and aerial mycelium on rich medium plates having glucose as carbon source (FIG. 1). Under the microscope, AW02 showed filamentous cells comprising peculiar droplets. These could be stained with the fluorescent dye Nile red (FIG. 2). This indicates intracellular storage of triglycerides. AW02 was capable of forming biomass in submerged culture on mineral salt medium. The best growth was measured with soya oil, the lowest with glycerol as carbon source. At most, 3 g/l of biomass were obtained from 5 g/l of substrate (see Table 1).

TABLE 1

| | Dry weight [g/l] | |
|---|---|---|
| Carbon source | Measurements | Mean values ? standard deviation |
| Glycerol | 0.130 | 0.1 ± 0.0 |
| | 0.104 | |
| | 0.094 | |
| Glucose | 0.996 | 1.3 ± 0.3 |
| | 1.238 | |
| | 1.642 | |
| Sucrose | 1.342 | 1.6 ± 0.9 |
| | 0.564 | |
| | 2.830 | |
| Soya oil | 0.152 | 1.9 ± 1.3 |
| | 2.594 | |
| | 3.096 | |

The table shows the respective growth of Phialemonium spec. and Acremonium strictum isolate AW02 on minimal medium with 5 g/l of different carbon sources in each case. For this, shake flasks having a volume of 500 ml with 50 ml medium where each inoculated with 10⁷ spores. After three to five days at 34° C. and 120 rpm, the mycelium was harvested by centrifugation or filtration, and lyophilised.

In submerged culture, the isolated fungus showed massive sporulation. In 500 ml shake flask culture, 10⁸ spores per ml were formed (see Table 2). In the phase contrast microscope, the spores appeared unicellular, presented often two noticeable intracellular particles and had a size of approximately 10 μm (FIG. 3).

TABLE 2

| | Spores [ml] | | |
|---|---|---|---|
| Glucose | Individual | | Standard |
| concentration | values | Mean values | deviation |
| 10 g/l | 4.60E+08 | 3.95E+08 | 6.50E+07 |
| | 3.30E+08 | | |
| 20 g/l | 3.40E+08 | 4.25E+08 | 8.50E+07 |
| | 5.10E+08 | | |
| 50 g/l | 7.50E+08 | 7.60E+08 | 1.00E+07 |
| | 7.70E+08 | | |

It is shown the respective sporulation of Phialemonium spec. and Acremonium strictum isolate AW02 on minimal medium for different glucose concentrations. 50 ml medium were inoculated in each of six 500 ml baffled flasks and incubated for three days at 34° C. The spores were counted in the Thoma chamber.

### Example 2

Establishment of a Simple Process for the Production of a Lipase

For the production of an inoculum, the fungus was incubated in a sterile minimal medium comprising 20 g/l glucose and an initial pH 3 at 34° C. After four days incubation of 100 ml in a 500 ml baffled flask, the mycelium had completely broken up into spores. The spores could be stored with 30% glycerol at −20° C. A 200-litre vessel comprising 100 litres of minimal medium was inoculated with these spores. Neither the vessel nor the medium were sterilised.

Progress:

Day 1 Inoculation with 100 ml sporulated preculture
Minimal medium with 5 g/l soya oil, pH 3, room temperature,
stirred 1× day using drilling machine and concrete agitator
Aeration 14 l/min compressed air through a 0.2 μm membrane filter

Day 4 Macroscopically: very slight medium turbidity
Microscopically: isolated mycelium filaments

Day 5 Macroscopically: growth visible compared to previous day
Microscopically: growth visible compared to previous day

Day 6 Macroscopically: clear growth visible compared to previous day, small mycelium balls
Microscopically: clear growth visible compared to previous day

Day 7 Macroscopically: clear growth visible compared to previous day
Microscopically: clear growth visible compared to previous day, formation of spores

Day 8 Macroscopically: growth visible compared to previous day, mycelium uniformly distributed in the entire medium
Microscopically: many spores

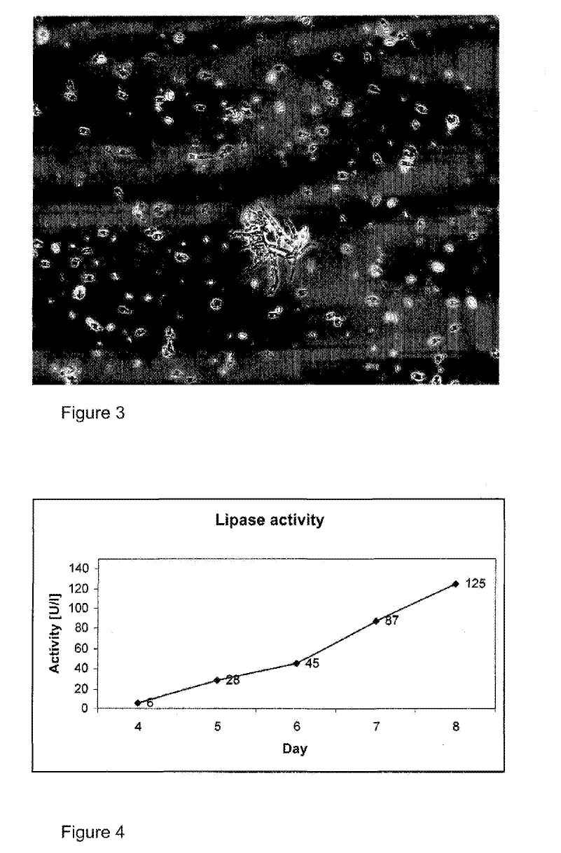

From the fourth day, lipase activity was measurable in mechanically homogenised samples. A maximum release of 125 μmol/l/min nitrophenol was measured from a palmitic acid ester (FIG. 4).

In order to examine the localisation of the lipase activity, 40 ml culture broth were centrifuged on the eighth day. 24 U/l were measurable in the supernatant. The pellet was resuspended in 5 ml water and homogenised. A lipase activity of 700 U/l was measured therein. Since this resulted in a reduction of the volume to one eighth, this means that the predominant part of the activity was cell-associated.

Out of 500 ml oil used for 100 l medium, after termination on the eighth day, 460 ml could still be skimmed as lighter phase from the culture broth surface.

### Example 3

Influence of the Medium on Lipase Production

It could be shown that changes in the carbon source and energy source or in the salt concentrations have a clear impact on the growth and the production of lipase activity. The use of deionised water could not be dispensed with. Using tap water, Phialemonium spec. and Acremonium strictum, respectively, practically did not grow. The use of glucose only led to very low lipase activities.

Although the lipase activity can be measured in accordance with the standard literature lipase assay (Okeke and Okolo, supra), a new continuous lipase assay, according to Kabaoglu, having a constant pH value of 8.0, was established, in which desoxycholate was exchanged against Triton X-100. The reliability of the assay was checked using four commercially available lipases (see Table 3), and good agreement was obtained with the manufacturer's specifications.

TABLE 3

| | | Indicated Activity | Demonstrated |
|---|---|---|---|
| Manufacturer | Designation | [U/ml] | Activity [U/ml] |
| Novostab | K 311305i | 12,000 | 12,928 |
| Novarenko | K 311307ik | 20,000 | 18,808 |
| Novarenko | K 311307i | 5,000 | 5,885 |
| | Saulich | — | 1.140* |

*in [U/mg]

Assay check by the analysis of four commercially available lipases. The assay was carried out with 100 μl pure enzyme solution.

Furthermore, the influence of soya bean oil or glucose in minimal medium on the extracellular lipase activity was investigated. The highest activity (˜90 U/l) in the culture supernatant was demonstrated with 10 g/l soya bean oil. The lowest activity (˜1 U/l) was found in the case of growth on glucose (see Table 4).

TABLE 4

| | Activity [U/l] | | | | |
|---|---|---|---|---|---|
| | Day 5 | Day 6 | Day 7 | Day 8 | Day 11 |
| 10 g/l soya bean oil | 88 | 14 | n.d. | 1 | n.d. |
| | 68 | 15 | n.d. | 1 | n.d. |
| 20 g/l soya bean oil | 5 | n.d. | n.d. | 1 | n.d. |
| | 9 | 4 | 31 | n.d. | 5 |
| 50 g/l soya bean oil | 14 | 10 | 6 | 4 | 2 |
| | 28 | 7 | n.d. | n.d. | 5 |
| 20 g/l glucose | 1 | n.d. | n.d. | n.d. | n.d. |
| | 1 | n.d. | n.d. | n.d. | n.d. |

Lipase activity in culture supernatants. The strain AW02 was cultured in minimal medium with 1.5 g KNO₃, 0.5 g MgSO₄×7H₂O, 1.5 g KH₂PO₄, 0.5 mg FeSO₄×7H₂O, 0.5 mg ZnSO₄×7H₂O, 0.02 mg CuSO₄×5H₂O and 0.02 mg MnCl₂, dissolved in 1 l distilled water. Soya bean oil or glucose was used in different concentrations as the only carbon source. The experiment was carried out as double determination in two independent shake flasks. The cultures were incubated at 32° C. and 120rpm, and the assay was carried out as follows:

For the lipase determination, 2 ml of sample was taken from each culture, centrifuged for 15 min at 13,000 rpm, and the supernatant was used for the assay. A substrate mixture A/B (1:9, v/v) was prepared by emulsifying the solution A (30 mg p-nitrophenyl palmitate, dissolved in 10 ml isopropanol) in solution B (0.8 g Triton X-100, 0.1 g gum arabic in 100 ml 100 mM Tris-HCl pH 8.0). 900 μl of the mixture A/B were incubated with 100 μl enzyme solution, and the activity was determined over a period of 240 seconds at 405 nm and 30° C. Heat-inactivated enzyme (10 min, 95° C.) was measured as negative control. The values indicated as “n.d.” in the table were not detectable. The limit for this measurement process was <1 U/l.

After the harvest, the lipid activity in the culture supernatant and in the mechanically homogenised mycelium was determined. A 100-fold increased activity was found in the mycelium compared to the culture supernatant. This means that the lipase was cell-associated.

TABLE 5

| | Activity [U/l] | | |
|---|---|---|---|
| | | French press | |
| | Culture | Digestion of the | Culture broth |
| | supernatant | mycelium | (calculated) |
| 10 g/l soya bean oil | n.d.* | 838 | 84 |
| | n.d. | 995 | 99 |
| 20 g/l soya bean oil | n.d. | 338 | 34 |
| | 5 | 192 | 19 |

*n.d. = not detectable

Determination of lipase activity with homogenised mycelium after a culture period of 11 days. 15 ml culture solution were filtered, the mycelium was harvested and suspended in 1.5 ml assay buffer (Solution B). The mycelium was subsequently homogenised by passing it through a French press and centrifuged for 20 min at 13,000 rpm. 100 μl of supernatant were examined as described above.

After 5 days of growth, a complete transformation of the mycelium into spores was observed after the glucose had been consumed. At least 10⁸ spores per 100 ml were produced.

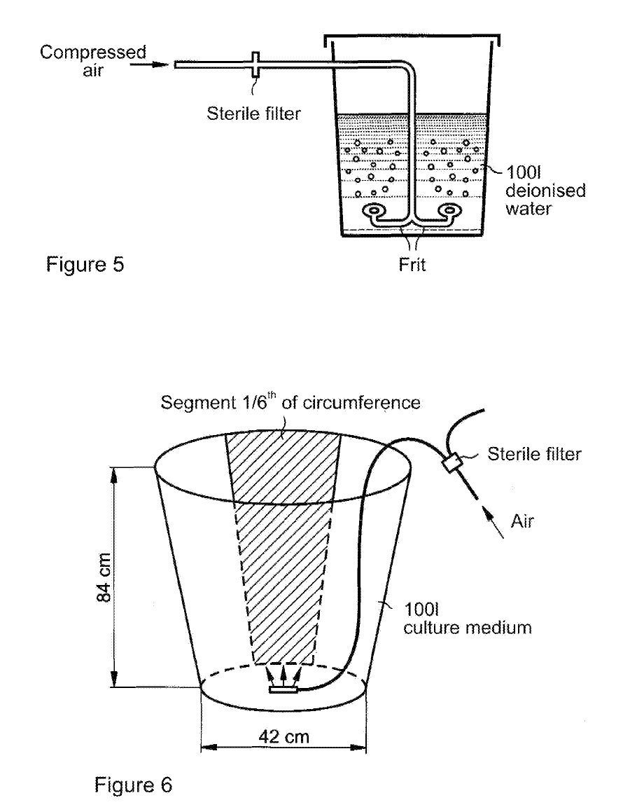

Furthermore, a 100 l culture was started. Even though it was not carried out under sterile conditions, no contamination with other microorganisms was observed. After 5 days of growth, 11 U/l, and after 6 days of growth, 7 U/l were detected in the culture supernatant. The culture device is shown schematically in FIG. 5. A culture vessel having a capacity of at least 100 l can be seen. The culture vessel is charged with mineral salt solution in deionised water. There is also shown a device for introducing compressed air, which reaches the frit of the vessel via a sterile air filter. The culture could be optimally aerated in this way.

A first approach to optimise the culture conditions began with a lipase activity of 5,700 U/l in the culture homogenate after 7 days of growth in 10 g/l soya bean oil. A reduction of the soya bean oil to 5 g/l showed an increase in activity to 6,200 U/l, and a doubling of the salt concentration showed an increase in activity to 9,000 U/l (see Table 6). No or much reduced growth was observed when tap water was used instead of distilled water. No lipase activity could be detected in tap water.

TABLE 6

| | Activity [U/l] | | | |
|---|---|---|---|---|
| | Day 2 | Day 3 | Day 4 | Day 7 |
| Distilled water, 10 g/l soya | 1 | 18 | 148 | 5,705 |
| bean oil, 1 × salt | 8 | 17 | 143 | 1,410 |
| Distilled water, 5 g/l soya | 62 | 275 | 2,445 | 6,206 |
| bean oil, 1 × salt | 23 | 119 | 1.668 | 6.286 |
| Distilled water, 10 g/l soya | 23 | 8 | 11 | n.d. |
| bean oil, 2 × salt | 24 | 118 | 834 | 8,958 |
| Tab water, 10 g/l soya bean | 8 | 10 | 4 | 11 |
| oil, 2 × salt | n.d.* | 1 | 6 | 3 |
| Tab water, 10 g/l soya bean | n.d. | n.d. | 7 | n.d. |
| oil, 1 × salt | n.d. | n.d. | 2 | n.d. |

*n.d. = not detectable

Determination of lipase activity in homogenised culture. 3 ml of culture were withdrawn from each of two different shake flasks, and the mycelium was homogenised by ultraturrax treatment for 1 minute. 100 μl were examined as described above.

### Example 4

Lipase Activity in the Biofilm

It could further be shown that Phialemonium spec. and Acremonium strictum AW02 respectively settle as biofilm on the surface of plastic segments which had been hung in a vessel, when no stirring is performed but rather only the rising air bubbles take care of convection. Over 99% of the enzyme activity was measured in the homogenised biofilm. Dispensing with the stirring eliminates the need for filtration or centrifugation for the cell harvest.

The objective of this experiment was to characterise the lipase activity of Phialemonium spec. and Acremonium strictum (strain AW02), respectively, in a 100 l culture. In particular, there was a need to examine the questions of whether the lipase is cell-bound or freely diffusing, and, in the case of the lipase being cell-bound, whether it can be removed and what temperature tolerance the lipase has.

Two vessels (see FIG. 6) were used for this experiment. FIG. 6 shows a vessel having the dimensions 84 cm×42 cm. It has a capacity of at least 100 l. Also shown is a device for introducing sterile air, with which the cell culture can be aerated. One of the vessels was cut vertically into six segments. Three of these segments were hung in the first vessel over the edge. By means of these segments, the biofilm forming on the surface could simply be pulled out of the medium and scraped off the surface.

The medium was inoculated with a spore suspension (100 ml, 10¹⁰ spores). Phialemonium spec. and Acremonium strictum AW02, respectively, grew under aerobic conditions in 100 l minimal medium with 1.5 g/l KNO₃, 0.5 g/l MgSO₄×7H₂O, 1.5 g/l KH₂PO₄, 0.5 mg/l FeSO₄×7H₂O, 0.5 mg/l ZnSO₄×7H₂O, 0.05 mg/l CuSO₄×5H₂O and 0.02 mg/l MnCl₂.

Phialemonium spec. and Acremonium strictum AW02, respectively, grew as a white biofilm on the surface of the segments of the vessel. The hyphens, which are embedded in the biofilm, were visible subsequent to staining with Congo red.

The biofilm was harvested following growth. Following suspension and centrifugation, a lipase activity test was carried out with both the supernatant and with the pellet, which was resuspended and homogenised by French press. The results showed that almost no lipase activity was detectable in the supernatant, while a lipase activity approximately 40 times greater than that of the supernatant was detectable in the suspension subsequent to the French press. This showed that the lipase is bound to the biofilm or cell-bound, as the case may be. In order to determine the lipase activity in the biofilm and the culture liquid, ⅙ (23.7 g) of the biofilm were harvested from the 100 l vessel. 1.5 g thereof were resuspended, homogenised by passing through a French press, and a lipase activity of 280 U/l measured therein. Moreover, 22.2 g were lyophilised, resulting in a dry weight of 3.2 g. 0.2 g thereof were resuspended, which resulted in a lipase activity of 920 U/l. In parallel, 200 ml culture medium were withdrawn from the 100 l vessel, concentrated to 20 ml by ultrafiltration, and subjected to a lipase assay. This lipase activity test resulted merely in 2 U/l. It could also be shown that the ultrafiltration only resulted in a weak increase in the activity of the culture medium, indicating that the lipase is released only to a small extent.

In order to determine the weak release of the lipase activity from the biofilm, a treatment with repeated shearing or glucanase treatment was used. One part of the biofilm was incubated with β-1,3-glucanase mixture (Glucanex) for 60 min at 32° C. The attempt to enzymatically degrade the matrix of the biofilm and/or the cell wall resulted in a weak release of the lipase activity into the supernatant. Another part of the biofilm was resuspended and treated with an ultraturrax for 1 min, 2 min and further 10 min. Following each treatment, the suspension and the supernatant were measured with respect to lipase activity. The lipase activity was released only slightly from the supernatant. Because of the loss of activity, the inventors suspect that the biofilm-bound lipase is destroyed by the ultraturrax treatment.

It was furthermore demonstrated that the lipase displayed a broad temperature tolerance. The biofilm was homogenised by passing it through a French press, and this homogenate was directly used for the stability test. This was followed by incubation at a temperature of from 30 to 65° C., each in 5-minute temperature steps for 60 min. The material was freeze-dried, and a lipase activity test was performed. The stability of the lipase activity was measured by one hour pre-incubation at temperatures of between 30° C. and 65° C. and subsequent activity test. No significant loss of activity was found between 30° C. and 45° C. After one hour at 50° C., the residual activity fell to 50%.

In summary, it could thus be established that Phialemonium spec. and Acremonium strictum, respectively, can be grown effectively as a biofilm in a vessel under non-sterile conditions. 99.4% of the lipase activity was bound to the biofilm. A weak lipase activity release was observed subsequent to mechanical shearing or glucanase treatment. The lipase activity was stable following storage at −20° C. and also following incubation for 60 min at 40° C.

In conclusion, it should be noted that all the features mentioned in the application documents and in particular in the dependent claims, regardless of the formal reference back to one or more specific claims, are also intended to be awarded protection in their own right individually or in any combination.

### REFERENCE LIST

Okeke and Okolo, (1990), “The Effect of Cultural Conditions on the Production of Lipase by Acremonium strictum”, Biotechnology Letters 12:747-750

Kabaoglu F (2005) Studien zur Optimierung der rekombinanten Genexpression in der methylotrophen Hefe Pichia pastoris, thesis, University of Constance, Biology Department.

We claim:

1. A submerged culture process for producing an enzyme, comprising the steps of
a) providing a minimal medium having deionised water, mineral salts and an organic carbon source, in a vessel,
b) inoculating the minimal medium with a fungus,
c) incubating the minimal medium at a pH value of 1-4 for a period of time which is sufficient for the fungus to be optically visible in the minimal medium, and
d) obtaining the enzyme,
wherein the minimal medium and the vessel are not sterilised, and the fungus is Phialemonium spec. and/or Botrytis cinerea.

2. The process according to claim 1, wherein the enzyme is selected from the group consisting of lipase, amylase and protease.

3. The process according to claim 1, wherein the carbon source is a fat, polysaccharides or proteins.

4. The process according to claim 3, wherein the fat is preferably a vegetable triglyceride, more preferably soya oil.

5. The process according to claim 1, wherein the minimal medium further compromises a nitrogen source, and the nitrogen source is nitrate.

6. The process according to claim 1, wherein the minimal medium contains, relative to one litre of water,
approximately 0.5 to 10 g KNO₃
approximately 0.5 to 5 g KH₂PO₄
approximately 0.2 to 0.75 g MgSO₄×7H₂O
approximately 0.2 to 0.75 g FeSO₄×7H₂O
approximately 0.2 to 0.75 g ZnSO₄×5H₂O
approximately 0.01 to 0.05 g CuSO₄×5H₂O
approximately 0.01 to 0.05 g MnCl₂×4H₂O and
approximately 5 to 100 ml soya oil.

7. The process according to claim 5, wherein the minimal medium contains, relative to one litre of water,
approximately 1.5 g KNO₃
approximately 1.5 g KH₂PO₄
approximately 0.5 g MgSO₄×7H₂O
approximately 0.5 g FeSO₄×7H₂O
approximately 0.5 g ZnSO₄×5H₂O
approximately 0.02 g CuSO₄×5H₂O
approximately 0.02 g MnCl₂×4H₂O and
approximately 18 ml soya oil.

8. The process according to claim 1, wherein the pH value is 3.

9. The process according to claim 1, wherein the incubation time is approximately 3 to 14 days.

10. The process according to claim 1, which is performed at a temperature of between approximately 20° C. and approximately 45° C., preferably at 20° C. to approximately 35° C., more preferably at approximately 21° C.

11. The process according to claim 1, wherein the minimal medium is aerated via a sterile filter.

12. The process according to claim 1, wherein the minimal medium is inoculated with a pure culture inoculum.

13. The process according to claim 1, wherein the fungus is harvested from a carrier material which is submerged into the minimal medium.

14. The process according to claim 1, wherein the volume of the minimal medium is approximately 1 litre to approximately 3,500 m³, preferably approximately 100 litres, more preferably approximately 350 litres.

15. An enzyme produced by the process according to claim 1.

16-17. (canceled)
